(19) United States
(12) Patent Application Publication
(10) Pub. No.: US 2010/0037353 A1
(43) Pub. Date: Feb. 11, 2010

(54) METHOD FOR IMPROVING ACTIVITY OF REACTIVE OXYGEN SPECIES SCAVENGING ENZYMES

(75) Inventors: Masayuki Suzuki, Chita-shi (JP); Kazunori Shinji, Chita-shi (JP); Kazuo Ogasa, Chita-shi (JP); Satoshi Makishima, Chita-shi (JP); Tatsuya Uraji, Chita-shi (JP)

Correspondence Address: OLIFF & BERRIDGE, PLC, P.O. BOX 320850, ALEXANDRIA, VA, 22320-4850, US

(73) Assignee: B Food Science Co., Ltd., Chita-shi (JP)

(21) Appl. No.: 12/450,729

(22) PCT Filed: Apr. 16, 2008
(86) PCT No.: PCT/JP2008/057399

§ 371 Date: Oct. 9, 2009

(30) Foreign Application Priority Data
Apr. 19, 2007 (JP) 2007-111046

(51) Int. Cl. A01H 5/00; A61K 31/045; A61K 31/70

(52) U.S. Cl. 800/298; 514/738; 514/23

(57) ABSTRACT

A highly safe, inexpensive and widely utilizable method, which has a mechanism backed by scientific grounds, for improving an activity of ROS (reactive oxygen species)-scavenging enzyme group; An increase in the amount of an enzyme or promotion of an enzyme activity of the ROS scavenging enzyme group such as superoxide dismutase, catalase or peroxidase is caused in an organism having the ROS-scavenging enzyme with one or two or more kinds of substances selected from the group consisting of erythritol, mannitol, sorbitol and xylitol in 0.01-10% administration concentration.

### TECHNICAL FIELD

The present invention relates to a method to improve a reactive-oxygen-species (ROS)-scavenging enzyme's activity which is lowered by a lifestyle such as smoking, aging, illness or disease, psychological stresses etc. The enzyme's activity is improved by a mechanism in which the enzyme is induced, expressed and strengthened by erythritol, mannitol, sorbitol and xylitol. In addition, the present invention relates to a method for supplementing prevention and/or treatment of the ROS-related diseases.

### BACKGROUND ART

Organisms do aerobic respiration (or oxygen breathing) in order to obtain energy essential for living by decomposing ingested organic matter. Through the aerobic respiration, the organisms can obtain energy, but at the same time they have a risk of being constantly exposed to oxidative stress. This oxidative stress is caused by the ROS which has an unpaired electron in an oxygen molecule and accordingly has high reactivity.

Oxygen-breathing organisms have the ROS-scavenging enzyme group in their body as a mechanism to scavenge the ROS. Generally, the concentration of the ROS is kept extremely low due to the presence of the ROS-scavenging enzyme. However, when the level of the ROS production rate relatively surpasses the level of the activity of the ROS-scavenging enzyme due to a lifestyle such as smoking, aging, illness or disease, psychological stresses etc., the balance between these two levels becomes disrupted. The generated excessive ROS causes injury to cells, thus accelerating aging processes or directly causing various types of diseases such as cancers or lifestyle-related diseases.

As the ROS-scavenging enzyme, superoxide dismutase (SOD) (enzyme code (EC) 1.15.1.1), catalase (EC 1.11.1.6), peroxidase (EC 1.11.1.7), glutathione peroxidase (EC 1.11.1.9) etc. can be exemplified. However, their details vary with species of the organisms. In the case of superoxide dismutase, for example, it is known that rats and humans have 3 types 3 species of SOD isozymes: Cu/Zn-, Mn- and EC-SOD isozymes and Arabidopsis thaliana has 3 types 8 species of SOD isozymes: Cu/Zn-, Mn- and Fe-SOD isozymes.

Recently, relationship between the SOD activity and the development of diseases has been spotlighted. For example, it was reported that Werner syndrome and amyotrophic lateral sclerosis occur when the SOD activity drops. Further it is reported that diabetics have a low SOD activity due to the Maillard reaction because the Maillard reaction remarkably causes a decrease in the SOD activity (Non-Patent Document 1, shown below). Furthermore, when the SOD activity drops, various complications are induced because protective capacity to the oxidative stress is lowered.

As a method for treating the diseases caused by the ROS, such a method that captures the excessive ROS in the organism so as to eliminate the cytotoxicity of the ROS or a method that improves the activity of the ROS-scavenging enzyme system so as to scavenge the ROS in the same fashion as in a healthy, normal condition, is effective.

As a method for eliminating the cytotoxicity of the ROS by capturing the excessive ROS in the organism, ingestion of antioxidants typified by such as bilirubin, uric acid, vitamin E, carotenoids, isoflavonoids, phenols, ascorbic acids, etc. is disclosed (Tetsuo Nagano, Masahiko Mashino, Free Radical: Scavenger used for study of active oxygen and its characteristics, FARUMASHIA Vol. 27, p. 231-233 (1991), shown below). These antioxidants are substances which are oxidized more easily than the cells which are also susceptible to oxidative damage. The antioxidants are called as radical scavengers and react preferentially to the ROS as what is called, a substitute for the cell, to eliminate the cytotoxicity of the ROS. Foods that contain these antioxidants are so-called, SOD-like foodstuffs and acquire a wide support from consumers and are sold very well. The antioxidants have high reactivity, therefore they are unstable. During preservation, the antioxidants are subjected to chemical changes such as oxidation, decomposition etc. which are caused by the presence of oils and fats in the SOD-like foodstuffs. Accordingly, it is difficult to keep the effective amount of antioxidants in the foodstuffs by the time of ingestion. Further, the antioxidants can not preferentially and certainly eliminate the cytotoxicity of the ROS even when the antioxidants are taken into the organisms, because various chemical reactions described above have already proceeded. In addition, these antioxidants change themselves into a new radical species when they capture the ROS because of the mechanism of the chemical reaction, accordingly the cytotoxicity of the ROS can not be completely eliminated.

On the other hand, it has been proposed that sugar alcohols such as mannitol, sorbitol and xylitol which are chemically stable can be used as a hydroxyl radical (HO.) trapping agent (Patent Document 1, shown below). Similarly, it has been found that erythritol has a function of a radical scavenger with respect to the ROS such as a hydroxyl radical, a superoxide anion (O²⁻), a hydroperoxy radical (HOO.), a lipid peroxy radical, a lipid alkoxy radical etc., and it has been proposed that the erithritol can be used as foodstuffs, foodstuff additives, cosmetics and medicines for prevention and/or treatment of diseases (Patent Document 2, shown below). However, availability of these series of sugar alcohols as a radical scavenger has been demonstrated only in vitro but there is actually no direct in vivo evidence for scavenging of the ROS and eliminating of the cytotoxicity, and the action mechanism of these alcohols has not yet been clarified.

As a method for improving the activity of the ROS-scavenging enzyme system and thus scavenging the ROS in the same manner as in a healthy, normal condition, ingestion of extracts of various crude drugs (Japanese Patent Publication JP 2002-29992, shown below), extracts of Ganoderma lucidum group mushrooms (Japanese Patent Publication JP 2002-173441, shown below), and tocopheryl phosphate (Japanese Patent publication JP 2006-232767, shown below), all of which have an activity promotion effect for the ROS-scavenging enzyme system specifically for SOD and catalase, is disclosed. Unlike the aforementioned method in which the cytotoxicity of the excessive ROS in the organism is directly eliminated by the antioxidants, the ROS-scavenging enzyme's activity-promotion substance in the extracts of the various crude drugs and the Ganoderma lucidum group mushrooms acts as a trigger substance which promotes the SOD and catalase activities and has a feature to indirectly scavenge the ROS using a small amount of the promotion substance. These extracts of the various crude drugs and the Ganoderma lucidum group mushrooms can be obtained through extraction procedures from edible and ingestible natural products. Therefore, the extracts are considered to be harmless and safe and can be widely used for foodstuffs based on our food experience. However, the effective amount of the active substances to facilitate the activity of the ROS-scavenging enzyme system has not been determined and the mechanism of the action is unknown. Furthermore, the content of the active substances varies with harvesting place and time, therefore can not be fixed. Accordingly, it is difficult to keep a constant drug formulation. On the other hand, unlike the extracts of the various crude drugs and the Ganoderma lucidum group mushrooms, the active substance of tocopheryl phosphate has already been identified and its molecular structure has also been clarified. Accordingly the mechanism of the activity promotion of the ROS-scavenging enzyme system can be calculated. However, safety of tocopheryl phosphate is not secured in terms of the synthetic production process and food experience. Actually, as shown in Examples of Patent Document 5, its use is obliged to be limited to cosmetics such as cream.

When compared with the method for scavenging the ROS using the aforementioned antioxidants, the method for scavenging the ROS by improving or promoting the activity of the ROS-scavenging enzyme system is excellent in its action mechanism because the ROS is finally led into water and oxygen and detoxified. However, there have been only a few reports on such substances which have the activity promotion effect, and no substance is known whose action mechanism is clarified and safety is secured. Therefore there is no such substance which can be widely used for foods and beverages.

### DISCLOSURE OF THE INVENTION

The present invention was made to solve such problems mentioned above and an object of the present invention is to provide a high safety method for improving the activity of a reactive-oxygen-species-scavenging enzyme group which has an action mechanism backed by scientific grounds and can be used widely and inexpensive.

In order to solve the problems mentioned above, transcriptomics and metabolite analysis was carried out using a DNA microarray as a screening method to search and evaluation of substance that induces an increase in the enzyme amount or the promotion of the enzyme activity of the ROS-scavenging enzyme group.

The DNA microarray is a partial sequence of DNA arranged and fixed in a high-density manner on a slide glass which is divided into from several tens of thousands to hundreds of thousands. By using this, an expressed gene can be detected. Arabidopsis thaliana and a rat were used as model organisms. These organisms each was cultivated or bred with an arbitrary test substance and then an mRNA extracted from the cells of the cultivated or bred organism was hybridized with a target which is changed into cDNA with a reverse transcriptase. By carrying out this procedure, gene information expressed in the model organism was detected comprehensively, this procedure is so-called a transcriptomics. Furthermore, separation and purification of the protein of the ROS-scavenging enzyme in the model organisms' cells and determination of the enzyme activity were carried out, whereby the results obtained from the transcriptomics were directly verified.

RNA was extracted from a root of the Arabidopsis thaliana grown by a culture medium which contains a MS culture medium composition having sucrose at a concentration of 2% and a series of test substances at a final concentration of 0-2% each. This extracted specimen was supplied to the DNA microarray. In addition, RNA was extracted from the hepatocytes of SD rat grown with feed which contains a control feed having cornstarch as the main ingredient and a sugar alcohol as a series of test substances at a final concentration of 0-10%. This extracted specimen was supplied to the DNA microarray in the same way as described above. The protein of the ROS-scavenging enzyme in the model organism cells was separated and purified using AKT Aexplorer (FPLC). SOD activity measurements were carried out according to a standard protocol using SOD Assay Kit-WST.

As a result of the DNA microarray, data that indicate the induction of the ROS-scavenging enzyme was obtained from the specimens that were fed with a sugar alcohol such as erythritol, mannitol, sorbitol and xylitol. In particular, the result of the specimen which was fed with erythritol showed that SOD was strongly induced. That is, the mechanism of the ROS scavenging in the presence of a sugar alcohol such as erythritol is not the one that detoxifys the ROS using a radical scavenger such as a publicly-known antioxidant but the one that promotes the activity of the ROS-scavenging enzyme. The sugar alcohol such as erythritol etc. has the same effect as that of the extracts of the various crude drugs or Ganoderma lucidum group mushrooms or tocopheryl phosphate, but the mechanism is different in that it promotes the activity of the ROS-scavenging enzyme by inducing the ROS-scavenging enzyme itself. Accordingly, the sugar alcohol such as erythritol etc. expresses a completely new ROS-scavenging mechanism which has not yet been known. Further, when the sugar alcohol such as erythritol etc. was used, it was found by metabolomic analysis that the ROS-scavenging enzyme group was detected at a higher level than the control. Furthermore, the observed high ROS-scavenging activity value also supports the aforementioned induction of the ROS-scavenging enzyme. By the way, Patent Documents 1 and 2 disclose that erythritol, mannitol, sorbitol and xylitol themselves capture the ROS and accordingly eliminate the citotoxicity of the ROS. There is no description or suggestion about that erythritol induces the increase in the amount and the activity of the ROS-scavenging enzyme group, especially of the SOD.

The present inventors completed the present invention based on the results mentioned above in which erythritol, mannitol, sorbitol and xylitol induce the increase in the amount of an enzyme or the promotion of the enzyme activity of the ROS-scavenging enzyme group.

That is, the present invention is to provide a method for inducing an increase in the amount of an enzyme or a promotion of the enzyme activity of the ROS-scavenging enzyme group, which comprising a step of administering at least one of a sugar alcohol selected from a group consisting of erythritol, mannitol, sorbitol and xylitol to an organism such as for example a human, non-human animal, plant etc. having the ROS-scavenging enzyme group. Further, the present invention is to provide a method for supplementing prevention and/or treatment of ROS-related diseases by inducing the increase in the amount of an enzyme or the promotion of an enzyme activity of the ROS-scavenging enzyme group. Still further, the present invention is to provide an organism such as an animal or plant having an antioxidative potential given by inducing the increase in the amount of enzyme or the promotion of an enzyme activity of the ROS-scavenging enzyme group. The above mentioned substances are useful as pharmaceutical ingredients such as an enzyme-amount-increasing promoter for the ROS-scavenging enzyme group, an enzyme-activation-promoting agent for the ROS-scavenging enzyme group. These substances are also used as additives for foodstuffs, feedstuffs, fertilizers etc., active substances for drugs and medicines, supplementary ingredients for drugs and medicines, ingredients for cosmetics etc.

According to the present invention, the activity of a series of enzyme group, which efficiently and effectively eliminates the ROS which is said to be responsible for various diseases, can be induced. Therefore, the present invention can be used for an easy-to-use and risk-free preventive method, medical treatment and a supplementary method to lower disease risk of the organisms including humans. In addition, according to the present invention, SOD-like foodstuffs and food ingredients can be provided, and an antioxidant potential of agricultural and marine products including animals such as farm animals, poultries etc.; cereal plants such as rice plants, wheat plants etc.; vegetation such as plants, vegetables, fruit trees etc. can be improved. Further, sugar alcohols such as erythritol etc. used in the present invention are useful as drugs and additives.

The best mode of the present invention will be explained hereunder but it is noted that the scope of the present invention is not limited to these embodiments.

In the present invention, a sugar alcohol such as erythritol, mannitol, sorbitol and xylitol is administered into the organisms. In the administered organisms, an increase in the amount of the ROS-scavenging enzyme group, especially an increase in the amount of the ROS-scavenging enzyme or a promotion of enzyme activity can be induced. When an object of the present invention is to supplement prevention and/or treatment of a disease by inducing the enzyme in such a level that is effective for prevention and/or treatment of the disease caused by the ROS, the only thing to be done is to add the sugar alcohol such as erythritol, mannitol, sorbitol and xylitol into injectable solutions, drops, embrocations, adhesive preparations, daily foods or food ingredients or beverages. Similarly, when raising farm animals or poultries, these sugar alcohols are added to injectable solutions, feedstuffs or feedwater. In the case of glowing plants, these sugar alcohols can be added into foliar application agents, agrichemicals, spraying water etc. In this instance, concentration of erythritol, mannitol, sorbitol and xylitol in the foodstuffs, food ingredients, beverages, feedstuffs, feed waters, spraying water is preferably in the range of not less than 0.01% by weight, more preferably in the range of 0.05-10% by weight. When added amount is less than 0.1% by weight, effects of the increase in the amount of the ROS-scavenging enzyme or the promotion of the enzyme activity can not be fully expected. There is no limit in administration frequency, but in usual the frequency can be the same as that of usual food intake, water drinking, water spraying etc.

A method for adding erythritol, mannitol, sorbitol and xylitol, all of which have an effect on the increase in the amount of the ROS-scavenging enzyme or on the promotion of the enzyme activity and preparing an injection solutions, drops, capsule medicines, pills, suspensions, emulsions, powder medicines, tablets, syrup, lozenges, adhesive skin patches, forliar application agents, agricultural chemicals, foodstuffs, food ingredients, beverages, feedstuffs, feed waters, spraying waters etc. is not specifically limited. These sugar alcohols can be solely and directly dissolved, or can be made into a bulk form with other sugar or sugar alcohol and then dissolved into drugs. In addition, when the drugs are in the form of a solid body, the drugs are immersed into an aqueous solution of the sugar alcohol, or the drugs can be kneaded with diluents. The thus obtained drugs can be administered through oral or injection route as well as application and patching. Administration can be carried out by spraying etc.

In the present invention, erythritol, mannitol, sorbitol and xylitol can be used for foodstuffs or food ingredients to induce an increase in the amount of enzyme or a promotion of enzyme activity of the ROS-scavenging enzyme group. Because the safety of the substances of the present invention is secured, the present invention is excellent in environmental maintainability and in safety to the organisms especially to the human body. In addition, the present invention which is used to prevent and treat diseases caused by the ROS or to supplement prevention and treatment of the diseases. The present invention works under a new, unique mechanism backed by scientific grounds, and is an excellent method which shows remarkable effect even when a small amount of sugar alcohol is used. The present invention can be widely and inexpensively used in maintaining resistance characteristics against the ROS. The present invention can be utilized for health promotion of human beings and is useful for the healthy growth of animals and plants. Further, good growing condition of various kinds of organisms such as animals and plants can be achieved.

Next, the present invention will be explained more precisely by referring to Examples below.

### Example 1

Total RNA was extracted from the root of Arabidopsis thaliana grown by a culture medium containing a 2% sugar-containing MS culture medium composition and a series of sugar alcohols at a final concentration of 0-2% each. Transcriptomics was conducted using a Arabidopsis thaliana DNA chip (the number of loading genes: 30,480 genes, the number of probe/gene: 6, probe length: 60mer) of GeneFrontier Corporation. Results showed that in the erythritol-, mannitol-, sorbitol- and xylitol-added groups, many Cu/Zn-type SODs and SOD Cu Chaperone genes in the ROS-scavenging enzyme such as SOD were up-regulated, especially in the erythritol-added group.

### Example 2

Rats were fed for thirty days with a control diet containing 552.5 parts by weight of cornstarch, 250 parts by weight of casein, 35 parts by weight of mineral, 10 parts by weight of vitamin mixture, 2.5 parts by weight of choline bitartrate, 50 parts by weight of corn oil and 100 parts by weight of powdered cellulose, or feedstuffs containing the same composition as described in the control diet except that a portion of the powdered cellulose was replaced with the series of sugar alcohols so that the feedstuffs contain sugar alcohols at the final concentration of 0-10% each. The livers were taken out of the killed SD system rats and the total RNA was extracted from the hepatocytes, and transcriptomics was conducted using a rat DNA chip (the number of loading genes: 21,205, the number of probes per gene: 9, probe length: 60mer) of GeneFrontier Corporation. Results showed that in the erythritol-, mannitol-, sorbitol- and xylitol-added groups, many Cu/Zn-type SODs and SOD Cu Chaperone genes in the ROS-scavenging enzyme such as SOD were up-regulated, especially in the erythritol-added group.

### Example 3

The roots of the Arabidopsis thaliana grown in Example 1 were crushed and water-extracted and then freeze-dried. Then each of the dried extract was dissolved in the buffer solution of 0.1M calcium phosphate to obtain a specimen. The SOD activity was determined according to a standard protocol using SOD Assay Kit-WST (Dojin Kagaku Kenkyu-Sho). Results showed that when compared to the control group, the promotion of the SOD activity was seen in the erythritol-, mannitol-, sorbitol- and xylitol-added groups. In particular, the SOD activity of the erythritol-added group increased by 2-5 times.

### Example 4

The livers of the rats fed in Example 2 each was crushed and then extracted with normal saline solution, and then freeze-dried. SOD activity was determined according to the same way as described in Example 3. Results showed that when compared to the control group, promotion of the SOD activity was seen in the erythritol-, mannitol-, sorbitol- and xylitol-added groups. In particular, the SOD activity of the erythritol-added group increased by 2 times.

### INDUSTRIAL APPLICABILITY

In the present invention, an increase in the amount of an enzyme and a promotion of the enzyme activity of the ROS-scavenging enzyme group can be induced by adding erythritol, mannitol, sorbitol and xylitol etc. to organisms having an ROS-scavenging enzyme. Disease risk caused by the ROS can be lowered by letting both enzyme amount and enzyme activity of the ROS-scavenging enzyme group increase and promote. Erythitol, mannitol, sorbitol and xylitol have been safely utilized as foodstuffs and food ingredients, accordingly the present invention can also be used highly safely. The method of the present invention is also used to supplement the prevention and/or treatment of disease caused by the ROS. Safety of the method is secured, and the method works based on a new and unique mechanism backed by scientific grounds. Further, the method creates remarkable effects with a small amount of substance and is an excellent method and also can be widely used for promoting health and keeping good physical conditions of animals, vegetation and human beings at low cost. Therefore, the present invention can be widely used as foodstuffs, food additives, drugs, drug additives, cosmetics, agricultural chemicals, feed additives, and manure additives.

1. A method for inducing an increase of an enzyme amount or a promotion of an enzyme activity of reactive-oxygen-species (ROS)-scavenging enzymes, which comprising a step of administering at least one substance selected from the group consisting of erythritol, mannitol, sorbitol and xylitol in a concentration of 0.01-10% to an organism having a reactive-oxygen-species (ROS)-scavenging enzyme such as superoxide dismutase, catalase and peroxidase.

2. A method for supplementing prevention and/or treatment of disease by using the method according to claim 1,
further comprising a step of inducing the increase of the enzyme amount or the promotion of the enzyme activity of the ROS-scavenging enzymes in an organism to induce a level that is effective for prevention and/or treatment of a disease caused by the ROS.

3. An organism having an antioxidative potential which is prepared by a step of administering at least one substance selected from the group consisting of erythritol, mannitol, sorbitol and xylitol to the organism having the ROS-scavenging enzyme, and inducing an increase in an enzyme amount or a promotion of an enzyme activity of the ROS-scavenging enzymes.

4. A plant having an antioxidative potential which is prepared by a step of administering at least one substance selected from the group consisting of erythritol, mannitol, sorbitol and xylitol to the vegetation having ROS-scavenging enzymes, and inducing an increase in an enzyme amount or a promotion of an enzyme activity of the ROS-scavenging enzymes.
